(19) United States
(12) Patent Application Publication
(10) Pub. No.: US 2025/0037791 A1
(43) Pub. Date: Jan. 30, 2025

(54) ARTIFICIAL INTELLIGENCE (AI) IN SELF - NON SELF (SNS) MODELING IN TRIPLE NEGATIVE BREAST CANCER TO DEVELOP THIRD GENERATION IMMUNE CHECK POINT INHIBITOR

(71) Applicant: Kumarpal A. SHAH, Searingtown, NY (US)

(72) Inventor: Kumarpal A. SHAH, Searingtown, NY (US)

(21) Appl. No.: 18/751,664

(22) Filed: Jun. 24, 2024

Related U.S. Application Data
(60) Provisional application No. 63/516,499, filed on Jul. 29, 2023.

(51) Int. Cl. G16B 15/30; G16B 5/00; G16B 40/20; G16B 50/30; G16C 20/50

(52) U.S. Cl. CPC G16B 15/30; G16B 5/00; G16B 40/20; G16B 50/30; G16C 20/50

(57) ABSTRACT

Solutions for prophylaxis, immune therapy and vaccine strategies for triple negative breast cancer. The immune pathogenesis of cancer and its tumor micro environment (TME) is defined in terms of SNS concept that contributes to cancer drug resistance and metastasis. In one embodiment a method for identifying candidate drug compounds for treating cancer is provided by training an artificial intelligence engine for simulating Self-Non Self (SNS) modeling of normal subjects. An analysis module of an artificial intelligence engine is generated which directs the simulated SNS modeling to specific cancer to redefine cancer immune pathogenesis. SNS mimicking compounds are identified with the analysis module to target immune pathogenesis of cancer. The analysis module is applied to screen candidate cancer drugs that can be combined strategically with SNS mimicking compound for cancer therapy.

### CROSS-REFERENCE TO RELATED APPLICATIONS

This Application claims the benefit under 35 U.S.C. § 119 (e) from U.S. Provisional Patent Application No. 63/516,499 entitled Next Generation Immune Check Point Inhibitor for Cancer Immune Therapy and Vaccine filed on Jul. 29, 2023.

### BACKGROUND OF THE INVENTION

### 1. Field of the Invention.

The invention relates to artificial intelligence (AI) in Self-Non Self (SNS) modeling in triple negative breast cancer to develop third generation immune check point inhibitor for cancer immune prophylaxis, vaccine and therapy. More particularly, the invention is directed to the application of AI to triple negative breast cancer drug resistance and metastasis and related accelerated mortality. The method to overcome these problems in a predictable way with safety and efficacy with third generation immune check point inhibitor is detailed.

### 2. The Prior Art

The immune system is the host defense system that plays an important role in protecting us from cancers. Experts have divided the working of the immune system into early (Innate) immune system and delayed (Adaptive) Immune system. Nobel Laureates, Experts and drug companies are focused on the role of T and B cells in delayed or adaptive immune system and how it can be programmed for cancer cell death.

According to their concept, under normal conditions, the immune system performs a series of steps which lead to an anticancer immune responses and cancer cell death. These steps can be described as cancer-immunity cycle Chen D and Mellman I., Oncology meets immunology: the cancer-immunity cycle, Immunity 39, 1-10 (2013). The critical steps are:
Tumor cells produce mutated antigens that are captured by dendritic cells.
The dendritic cells prime T cells with tumor antigen and stimulates the activation of cytotoxic T cells.
Activated T cells then travel to the tumor and infiltrate the tumor environment.
The activated T cells recognize and bind to the cancer cells.
The bound effector T cells release cytotoxins, which induce apoptosis (Cell Death) in their targeted cancer cells.

Cancer cells can exploit these immune checkpoints as a way to evade immune detection and elimination. Thus, for example, PD-1 (Programmed Cell Death-1) is an immune check point that are expressed on the surface of activated T cells. These receptors interact with its ligand PD-L-1 and PD-L-2 that are expressed on the surface of dendritic cells or macrophages. PD-1 and its ligand PD-L-1 and PD-L-2 belong to family of immune check point proteins that act as co-inhibitory factors that can halt or limit the development of T cell Responses. The PD-1/PD-L-1 interaction ensures that the immune system is activated only at the appropriate time in order to minimize the host cell damage. This is done as under. In cancers, the PD-1/PD-L-1 pathway presents an adaptive immune resistance mechanism exerted by tumor cells in response to endogenous immune anti-tumor activity. PD-L-1 is over expressed in tumor cells or on non-transformed cells in tumor micro environment (Pardoll D. M., The blood of immune check points in cancer immune therapy, Nat Rev Cancer 12, 252-264 (2012). PD-L-1 expressed on the tumor cells binds to PD-1 receptors on the activated T cells, which leads to the inhibition of the cytotoxic T cells. These deactivated T cells remain inhibited in the tumor micro environment.

Cancer immune therapy, which reactivates weakened immune cells of cancer patients, has yielded great success in recent years. Among immune therapeutic agents, immune checkpoint inhibitors have been of particular interest and have gained approval by the FDA for treatment of cancers. Immune checkpoint blockade through targeting programmed cell death protein-1 (PD-1) has demonstrated promising anti-tumor effects in cancer immune therapy of many different solid and hematologic malignancies. However, despite promising results, a favorable response is observed only in a fraction of patients, recurrence is another potential problem. These aspects are reviewed by Makuku R and Khalili N et al in “Current and Future Perspectives of PD-1/PDL-1 Blockade in Cancer Immune Therapy” and published their findings in Journal of Immunology Research, Volume 2021, Article ID 6661406, 1-15 pages. In this paper, they have reviewed the current and future perspectives of PD-1/L1 blockade in cancer immunotherapy, with a particular focus on predictive biomarkers of response to therapy. They also discussed the adverse events associated with PD-1/L1/2 inhibitors, ranging from severe life-threatening conditions such as autoimmune myocarditis, mild and moderate reactions such as skin rashes, and adverse effects of monoclonal antibodies. They have explored the potential strategies for improving the efficacy of immunotherapy with PD-1/L1 checkpoint inhibitors.

In 2018 Nobel Prize in Physiology or Medicine was awarded to Tasuku Honjo and James Allison for their discoveries in cancer immunology that led to above understandings. Professor Honjo was awarded for his discovery of the programmed death molecule-1 (PD-1) on T cells.

Professor Allison discovered another important immunosuppressive molecule: cytotoxic T-lymphocyte antigen-4 (CTLA-4). Suppression of T cell activation by PD-1 and/or CTLA-4 is considered one of the major immune escape mechanisms of cancer cells. (Huang Pei-Wei and Wen-Cheng Change John, Immune checkpoint inhibitors win the 2018 Nobel Prize, Biomed J. 2019 October; 42 (5): 299-306).

The above discovery stimulated the industry search for the development of monoclonal antibodies. It has facilitated the working of the host immune system to overcome cancer's ability to resist the immune responses. It stimulates the body's own immune mechanisms to fight against cancer cells. Monoclonal antibody therapies against PD-1 and PD-L-1 are being routinely used in cancer therapies. Thus, for example, Nivolumab, an anti-PD-1 drug developed by Bristol-Myers Squibb. This is approved for previously treated metastatic melanoma and squamous non-small cell lung cancer. Pembrolizumab, developed by Merck is approved for previously treated metastatic melanoma. There are several other immune therapy options being used or are in the development stages. The efficacy of the immune checkpoint blocked with monoclonal antibodies in cancer treatment are remarkable.

However, not all patients respond to a single therapy.

To enhance and broaden tumor activity of immune check point inhibition, the next step in evaluation is combining agents with synergies mechanisms of actions.

An example of such combination therapy is the combination of PD-1/PD-L-1inhibition blocked with complementary check point inhibitor CTLA-4 in Melanoma and in non-small cell lung cancer (Ott P.A. et. al., Combination immune therapy: a road map, J. Immuno Ther Cancer 5:16, 2017).

Another approach used is Adaptive T cell therapy. This involves first isolating tumor-specific T cells from patients and the expanding them ex vivo. The tumor-specific T cells can then be infused into patients to give their immune system the ability to overwhelm remaining tumor cells. T cells can be harvested either from the patient's tumor (tumor infiltrating lymphocytes, TILs) or peripheral blood (peripheral blood lymphocytes, PBLs). Tumor specificity must be induced in PBLs either through antigen specific expansion or genetic engineering (Perica K et al., Adaptive T cell immunotherapy for cancer, Rambam Maimonides Med J 6 e0004, 2015).

After expansion in culture, tumor-specific T cells can be re-infused into the cancer patient.

A third approach developed for adaptive cell therapy is called as CAR-T cell therapy.

Here T cells are engineered to express chimeric antigen receptors (CARs) that recognize cancer specific antigens. This means researchers can prime the cells to recognize and kill tumor cells that would otherwise escape immune detection (Grupp S et al, Chimeric antigen receptor-modified T cells for acute Lymphoid leukemia, N. Engl J Med 368, 1509-1518, 2013) and kill tumor cells). CAR-T cells and T cells with engineered tumor-specific TCRs show anti-tumor activity in some solid tumors and hematological malignancies.

To summarize, The PD-1/PD-L1 Pathway represents an adaptive Immune resistance mechanism. This allows tumor cells to evade host immune cytotoxic responses. This problem has been successfully targeted with Anti PD-1 drugs, combination therapies and CAR-T therapy for multiple cancers. However, results are variable and not satisfactory (Makuku R et al, Current and Future Perspectives of PD-1/PDL-1 Blockade in Cancer Immunotherapy, J Immunol Res. 2021; 2021:6661406).

Therefor new advances are needed that can be rapidly applied for cancer therapies.
A. Glioblastoma Multiformes: Saxena R, Gottlin EB, Campa MJ, Bushey RT, Guo J, Patz EF Jr. and He Y-W (2024), details a second generation of immune check point inhibitor targeting Factor H with Factor H antibody. “Complement factor H: a novel innate immune checkpoint in cancer immuno therapy” in Front. Cell Dev. Biol. 12:1302490. doi: 10.3389/fcell.2024.130249. In the article, the importance of Factor H as an innate immune molecule and its role in immune evasion and immune suppressive molecule in cancer is detailed and developed a monoclonal antibody of Factor H as a Second generation immune check point inhibitor. The potential adverse effects of Factor H antibody that may lead to development of life threatening Atypical Hemolytic uremia syndrome (aHUS), adverse effects of monoclonal antibodies and its impact on host cells is also outlined.
B. Junnkkala S. et al studied “Exceptional resistance of human H2 Glioblastoma cells to complement-mediated killing by expression and utilization of Factor H and Factor H like protein 1” and published their findings in J Immunol. 2000; 164 (11): 6075-81.

Kumar S et al., Targeting PD-1/PD-L1 in cancer immunotherapy: An effective strategy for treatment of triple negative breast cancer (TNBC) patients, Genes & Diseases, doi.org/10.1016/j.gendis.2022.07.024, the entire contents of which are incorporated herein by reference thereto. The study describes maintaining the balance between eliciting immune responses against foreign proteins and tolerating self-proteins is crucial for maintenance of homeostasis. The functions of programmed death protein 1 (PD-1) and its ligand programmed death ligand 1 (PD-L1) are to inhibit immune responses so that over-reacting immune cells does not cause any damage to its own body cells.

F. Raheem, Antibody Drug Conjugates, dx.doi.org/10.5772/intechopen.110804,the entire contents of which are incorporated herein by reference thereto. The study describes how Antibody drug conjugates (ADCs) continue to change the treatment paradigm of breast cancer and recent regulatory approvals of next generation ADCs are shifting how breast cancer is classified and treated. ADCs combine precision targeting with traditional cytotoxic treatment allowing for the delivery of highly potent chemotherapeutic agents to malignant cells.

Breast Cancer: Smolag K et al, Complement Factor Expressed by breast cancer cells differentiates CD14+ human monocytes into immuno suppressive macrophages, Onco-immunology 2022, Vol. 9, No. 01, e1731135, the entire contents of which are incorporated herein by reference thereto. The study details how factor H expression in Breast cancer shortens survival, increases recurrence and lead to accelerated mortality.

Monteran, L., Chemotherapy-induced complement signaling modulates immunosupression and metastatic relapse in breast cancer, Nature Communications (2022) 13:5797 (doi.org/10.1038/s41467-022-33598-x), the entire contents of which are incorporated herein by references thereto. The study describes Mortality from breast cancer is almost exclusively a result of tumor metastasis and resistance to therapy and therefore understanding the underlying mechanisms is an urgent challenge. Chemotherapy, routinely used to treat breast cancer, induces extensive tissue damage, eliciting an inflammatory response that may hinder efficacy and promote metastatic relapse.

Alvarez-Lorenzo, S., Membrane-Bound Complement Regulatory Proteins in Breast Cancer: Are They Best Therapeutic Targets?, dx.doi.org/10.5772/intechopen. 109945, the entire contents of which are incorporated herein by reference thereto. The study describes how in breast cancer the membrane-bound complement regulatory proteins (mCRP) have been proposed as biomarkers of malignant cellular transformation. These are molecules capable of inhibiting therapeutic efficacy, from both antibodies and cytotoxic drugs.

Kim SM et al., The nature of triple-negative breast cancer classification and antitumoral strategies, Genomics Inform 2020;18(4):e35, doi.org/10.5808/GI.2020.18.4.e35, the entire contents of which are incorporated herein by reference thereto. The study describes how breast cancer is one of the most common cancers in women and is a leading cause of cancer related deaths. In 2018, approximately 266,000 women were expected to be diagnosed with invasive breast cancer in the United States, and 40,920 women were expected to die from it. Triple-negative breast cancer (TNBC), which accounts for 15% of all breast cancers, is characterized by the lack of estrogen receptor (ER), progesterone receptor (PR), and human epidermal growth factor receptor 2 (HER2) expression. TNBC often occurs in under 40-year-old women and has a mortality rate of 40% within the first 5 years after diagnosis, a median survival time after metastasis of only 13.3 months, and a recurrence rate after surgery of 25%. The treatment of such breast cancer fails to respond to hormone therapy directed to ER and PR and or HER2 treatment with Trastuzumab. TNBC generally occurs in younger women and compared to other types of breast cancer, it shows a high histologic grade, a high propensity to metastasize to distant organs and has a poor outcome with a high recurrence rate after adjuvant therapy, which mainly consists of systemic cytotoxic chemotherapy.

To summarize, TNBC is the most aggressive type of breast cancer and has a poor prognosis. Because it lacks the expression of hormone receptors and HER2 receptors, therapy is primarily based on systemic chemotherapy, rather than targeted agents. Although TNBC has a higher mutational load than other breast cancer types, it generally responds well to Immune check point Blocker (ICB) therapy. This led to FDA approval for the use of nab-paclitaxel in combination with atezolizumab in TNBC patients.

A careful perusal of the literature, poses a significant challenge is to identify new targets and associated biomarkers for the better therapy.

Below we describe third generation of immune check point inhibitor targeting immune pathogenesis of Self-Non Self path (SNS) path in triple negative breast cancer with SNS mimicking compound. The essential feature of this invention is that it targets new cancer immune pathogenesis that is directed to the host and its immune system.

The multi-dimensional immune pathogenesis of cancer with its host and its immune pathogenesis particularly with SNS modeling will contribute to novel way to overcome triple negative breast cancer resistance, metastasis and premature mortality. It will improve the safety profile of the patient. It is expected that AI application in describing this invention will allow predictive capabilities to judge safety and efficacy in triple negative breast cancer and will eliminate or overcome prior art problems of safety and efficacy in cancer therapies

The third generation of immune check point inhibitors with SNS mimicking molecule targets highly glycosylated Factor H that is over expressed in cancer cells. The immune suppression and immune evasion effects on TME by this molecule is a step proximal to the effects of first generation immune check point molecules. This is done by blocking Factor B, Factor D and Factor H that are involved in hyper amplifying the C3 amplification loop that contributes to immune evasion, immune suppression and host adverse effects of triple negative breast cancer.

Colon Cancer: Wilczek E et al, The possible role of factor H in colon cancer resistance to complement attack, Int. J. Cancer: 122, 2030-2037 (2008); The article shows generation of Cytotoxic immune responses with Factor H antibodies in vitro studies.

Lung Cancer: Ajona D et al, Down-regulation of Human Complement Factor H sensitizes Non-Small Cell lung cancer cells to complement attack and reduces in vivo Tumor growth, J Immunol 2007; 178-5991-5998; details method to down regulate Factor H with Antibodies to improve cytotoxic immune responses.

Pancreatic Ductal Cancer (PDAC): Speth C et al, Complement and fungal Dysbiosis as Prognostic Markers and Potential Targets in PDAC treatment, Current Oncol. 2022, 29, 9833-98545 details how hyper activation of complement system contribute to PDAC and Fungal infection.

Hematological Malignancies: Luo Shanshan et al, How Does Complement Affect Hematological Malignancies: From Basic Mechanisms to Clinical Application, REVIEW article, Front. Immunol., 29 Oct. 2020 Sec. Molecular Innate Immunity Volume 11-2020.

One critical advance in immune therapy was reported by Carolyn Bartuzzi who won Nobel Prize in 2022 (med.stanford.edu/news/all-news/2022/10/bertozzi-nobel.html). Carolyn Bertozzi showed that cancer cells are highly sugar coated. They use sialic acid-capped glycans to evade immune detection. Her company Palleon Pharmaceuticals for example is developing glyco immune drugs that work to remove that cloak in a mechanism analogous to that used by the approved checkpoint inhibitor therapies (cen.acs.org/people/profiles/Carolyn-Bertozzis-glycorevolution/98/i5).

In the Global Oncology Trends 2024 report, the latest estimates indicate that the number of patients affected by cancer will increase dramatically through the middle of the century across all regions of the world. Cancer incidence is expected to increase by 61% through 2050, reinforcing the critical need to drive consistency of care delivery, equitable access to care, as well as the need for continued improvements in available medicines. The report is cited as IQVIA Institute for Human Data Science, Global Oncology Trends 2024: Outlook to 2028 May 2024, the entire contents of which are incorporated herein by reference thereto.

According to above report, the global spending on cancer medicine increased to $223 billion in 2023 and is projected to rise to $409 billion by 2028. PD-1/PD-L1 inhibitors, which are widely used in solid tumor treatments, as first generation immune check point inhibitor accounts for $52 billion in spending in 2023, with expectations to increase to over $90 billion by 2028. The future of next-generation immune check point inhibitors in oncology is marked by clinical and commercial uncertainties, yet annual spending could potentially rise from $4 billion to $23 billion by 2028.

At the same time, the innovation ecosystem that discovers, develops, and delivers breakthrough therapies in entire new areas of science is operating near peak levels. Rising incidence, increasing numbers of treated patients, and the resulting expected growth in spending in this area will trigger an array of complex challenges over the next several years that are reflected in the trends highlighted in this report gives insight to the global oncology market.

Below we describe third generation of immune check point inhibitor targeting immune pathogenesis of Self-Non Self path (SNS) path in cancer with SNS mimicking compound. The essential feature of this invention is that it targets cancer immune pathogenesis that is directed to the host and its immune system.

Certain processes have been proposed to utilized AI in the development of drugs. One example is U.S. Pat. No. 11,462,304 entitled Artificial Intelligence Engine Architecture for Generating Candidate Drugs, the entire contents of which are incorporated herein by reference thereto. This Patent is directed to anticancer peptides and does not mention Self-Non Self (SNS) or immune pathogenesis. A second example is U.S. Published Patent Application 2020/0320363 entitled Artificial Intelligence Advisory Systems and Methods for Vibrant Constitutional Guidance, the entire contents of which are incorporated herein by reference thereto. This Application is directed to general guidance and drug selection and does not mention Self-Non Self (SNS) or immune pathogenesis. A third example is U.S. Published Patent Application 2021/0249108 entitled Artificial Intelligence Engine for Generating Candidate Drugs, the entire contents of which are incorporated herein by reference thereto. This Application is directed to classifying and comparing/contrasting drug properties and does not mention Self-Non Self (SNS) or immune pathogenesis. A fourth example is U.S. Published Patent Application 2021/0304854 entitled Artificial Intelligence Engine Architecture for Generating Candidate Drugs, the entire contents of which are incorporated herein by reference thereto. This Application is directed to classifying and comparing/contrasting drug properties and does not mention Self-Non Self (SNS) or immune pathogenesis. A fifth example is U.S. Published Patent Application 2021/0366581 entitled Artificial Intelligence Engine Architecture for Generating Candidate Drugs, the entire contents of which are incorporated herein by reference thereto. This Application is directed to generating a multi-dimensional representation of drug compounds and does not mention Self-Non Self (SNS) or immune pathogenesis.

The multi-dimensional immune pathogenesis of cancer with its host and its immune pathogenesis particularly with SNS modeling will contribute to novel way to overcome cancer resistance, metastasis and improve the safety profile of the patient. It is expected that AI application in describing this invention will allow predictive capabilities to judge safety and efficacy in cancer and will eliminate or overcome prior art problems of safety and efficacy in cancer therapy.

The third generation of immune check point inhibitors with SNS mimicking molecule targets highly glycosylated Factor H that is over expressed in cancer cells. The immune suppression and immune evasion effects on TME by this molecule is a step proximal to the effects of first generation immune check point molecules. This is done by blocking Factor B, Factor D and Factor H that are involved in hyper amplifying the C3 amplification loop that contributes to immune evasion, immune suppression and host adverse effects.

### SUMMARY OF THE INVENTION

The invention details prophylaxis, immune therapy and vaccine strategies for triple negative breast cancer. In one mode of invention, the new immune pathogenesis of triple negative breast cancer and its tumor micro environment (TME) is defined in terms of SNS concept that contributes to triple negative breast cancer drug resistance, metastasis and premature mortality.

The First mode of invention, is directed to the application of AI to the database of SNS to visualize detail sequences of immuno pathogenesis of triple negative breast cancer that involves a series of following steps

Step 1: Normal function of Factor H is to act as dominant immune regulatory molecule of host in both fluid and on host cell surface. Factor H is over expressed in breast cancer cells. This protects cancer cells as immune shield (Self protection of host immunity is hijacked).

Step 2: Normal function of Factor H is also to instruct the immune system to launch cytotoxic immune responses against intruders such as breast cancer cells. Over secreted Factor H on cancer cells directs the immune system to target cancer cells. But as an immune regulatory molecule, it inhibits or inactivates activated immune system that is directed against cancer cells (Non-Self attack by host Immunity is hijacked).

Step 3: This hijacking of Step 1-2 is auto amplified several folds through C3 amplification Loop. This hijacks the normal control of C3 amplification to induce cytotoxic immune responses (C3 amplification hijacked)

Step 4: Step 1-3 impairs cytotoxic function of host immunity leading to the breakdown of C3bB Convertase, a flagging molecule for cancer cells that is targeted for cytotoxic immune responses. This impairs down stream function of immune system to generate cytotoxic effector responses of immune system by hijacking effector immune system function-TME is now surrounded by Inflammatory molecules such as C3a-C5a, Cytokines and Neutrophils mediated inflammatory molecules. (Hijacking effector immune responses).

Step 5: Recycling of events in Step 1-4 contributes to aggressive behavior of cancer and makes it resistant to current modes of treatment such as surgery, radiation, chemotherapy and monoclonal antibodies (Therapy Resistance and metastasis).

The recommended chemotherapy in triple negative breast cancer amplify complement cascade and accelerated mortality.

In the second mode of the invention we detail a molecular method to target immune pathogenesis of triple negative breast cancer and its TME with SNS mimicking compound that neutralizes Step 1-5 outlined in the first mode of invention.

In 2A mode of invention the method is used to boost host immune responses as a prophylaxis in high risk cancer patients that have genetic background or it can also be used by normal people who wish to protect themselves against cancer.

In 2B mode of invention the method is targeted to patients with cancer to inhibit Step 1-5 as a Therapy of cancer

In 2C mode of invention, novel formulation methods are developed with current and evolving Drugs, vaccines and radiation methods by developing strategic combination with SNS mimicking molecule to Overcome cancer resistance and metastasis

Most specifically, the invention details sulfonic polymers and its novel formulation methods to inhibit SNS related immune pathogenesis of triple negative breast cancer and its related morbidity and mortality by targeting Factor H, Factor B and D.

AI approach is used in one mode of invention to predict and visualize immune pathogenesis of cancer as outlined in steps 1-5 of first invention,

AI is used in the second mode of invention for the screening and discovery of sulfonic polymers that have SNS Mimicking properties and its novel formulation methods as outlined in the second mode of invention.

AI is used in the third mode of invention to analyze SNS mimicking properties of sulfonic polymers so that it can predict human safety and efficacy before clinical trial.

AI in the fourth mode of invention is used to define immune pathogenesis of Cancer and its targeting with SNS mimicking compound to develop strategic novel formulation methods for clinical trials.

In one embodiment, the invention relates a method for identifying candidate drug compounds for treating cancer, including training an artificial intelligence engine for simulating Self-Non Self (SNS) modeling of normal subjects. Next, generating an analysis module of an artificial intelligence engine which directs the simulated SNS modeling to specific cancer to redefine cancer immune pathogenesis. Then, identifying SNS mimicking compounds with the analysis module to target immune pathogenesis of cancer and applying the analysis module to screen candidate cancer drugs that can be combined strategically with SNS mimicking compound for cancer therapy.

The method further includes storing genome data including data on over 30,000 human genes on a server, that is data on a plethora of human genes on a server. The applying step includes applying the analysis module to the stored genome data with a preferential bias for references to the 1 q32 chromosome and its protein. The applying step further includes applying the analysis module to the stored genome data with a preferential bias for references to the 1 q32 chromosome and its protein Factor H as a weak and vulnerable genomic location within the genome data contributing to cancer immune pathogenesis. The method further includes the steps of storing scientific literature documents on the server, wherein the scientific literature documents include over 17,000 articles (i.e. a plethora of articles) giving details of SNS abnormalities in different cancers and wherein the simulating step includes using natural language processing to extract protein data for simulating SNS modeling of proteins.

The SNS modeling of proteins are selected from the group consisting of Factor H, Factor D, and combinations thereof. The storing step further includes storing patient related characteristics including statistics, protocols and laboratory data, wherein the identifying step includes providing an automated robotic system of a second artificial intelligence engine for identifying SNS compounds from the patient related characteristics to target immune pathogenesis of cancer. The identifying step additionally includes identifying SNS mimicking compounds to target immune pathogenesis of cancer for prophylaxis and therapy. The candidate drug compounds include chemo therapy drugs, radiation, monoclonal antibodies, complement modifying drugs and vaccines. The applying step further includes recursively applying the analysis module to develop updated formulation strategies covering current and evolving cancer therapies, chemo therapy drugs, radiation, monoclonal antibodies, complement modifying drugs and vaccines.

In an alternate embodiment, the invention relates to a system for identifying candidate drug compounds for treating cancer. The system includes a processor and a server for storing a multiplicity of scientific literature documents, genome data and a tangible, non-transitory computer readable medium storing instructions. Execution of the instructions causes the processor to generate an analysis module of an artificial intelligence engine which utilizes natural language processing to extract clinical data and relationships. Execution of the instructions further causes the processor to apply the analysis module to the scientific literature documents to analyze drug compounds with a relationship to the genome data to identify candidate drug compounds that effect a 1 q32 chromosome position.

In a further embodiment, the invention relates to a device for identifying candidate drug compounds for treating cancer. The device includes a processor and a server for storing a multiplicity of scientific literature documents and genome data. The device further includes an analysis module of an artificial intelligence engine which utilizes natural language processing to extract clinical data and relationships from the scientific literature. A comparison module is provided to correlate the extracted clinical data from the analysis module to the genome data to identify candidate drug compounds that effect a 1 q32 chromosome position.

### BRIEF DESCRIPTION OF THE DRAWINGS

The advantages, nature, and various additional features of the invention will appear more fully upon consideration of the illustrative embodiments now to be described in detail in connection with accompanying drawings. In the drawings wherein like reference numerals denote similar components throughout the views:

FIG. 1 is a diagram illustrating an AI based integrated model of immunity in a normal host.

FIG. 2A is a second diagram illustrating AI based cancer piracy of Factor H in Glioblastoma Multiformes (GBM) as a first reference example.

FIG. 2B is a third diagram illustrating AI based cancer piracy of Factor H in Triple Negative Breast Cancer as a second reference example.

FIG. 3 is a chart of an AI based Self-Non Self related Immune pathogenesis of Cancer that contribute to immune resistance and metastasis with cancer.

FIG. 4 is a diagram illustrating an AI based SNS mimicking (sulfonic polymer) molecule.

FIG. 5 is a table reporting the results of in-vitro binding activity of SNS mimicking (sulfonic polymers) and complement proteins in serum.

FIG. 6 is a diagram illustrating an embodiment of an Artificial Intelligence (AI) tool kit used to develop Self-Non Self modeling for major life threatening cancers.

FIG. 7 is a diagram illustrating how third generation immune check point inhibitors limit cross-talk between tumor microenvironment and the immune system.

FIG. 8 is a flowchart depicting the various steps that are executed in the method according to one embodiment of the invention.

### DETAILED DESCRIPTION OF THE PREFERRED EMBODIMENTS

One object of the invention is to apply AI to the tool kit of SNS in cancer. Glioblastoma Multiformes is used as a reference example.

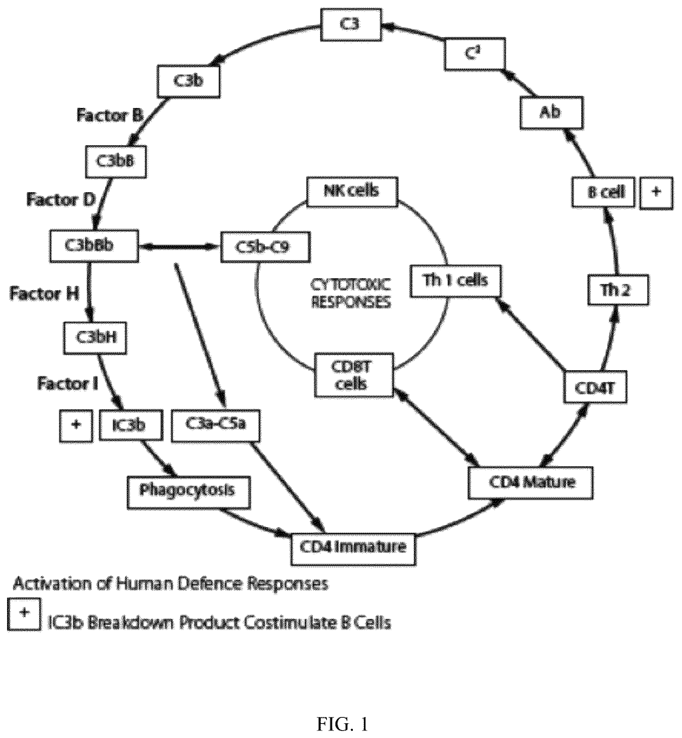

As shown in FIG. 1, AI approach is used to integrate complement based advances (C3: Classical, Lectin and Alternate Complement System) with delayed immune responses (T and B cell responses) as under.

C3 are three elements of Complement system where Classical and Lectin based system, join Alternate complement system to take part in C3 amplification process. This process is primarily driven by Factor B, Factor D and Factor H. The end objective of such C3 amplification system is to boost immune responses of host by the following steps.

First by forming C3bBb convertase, this acts as flagging molecule for intruder such as Cancer

Second by seeking cooperation from Factor H to form C5b-C9 cytotoxic immune responses so that stimulated immune system only target cancer cells.

Third, Factor H binds to highly glycosylated molecules of host cell and provide protection against adverse effects of stimulated immune system.

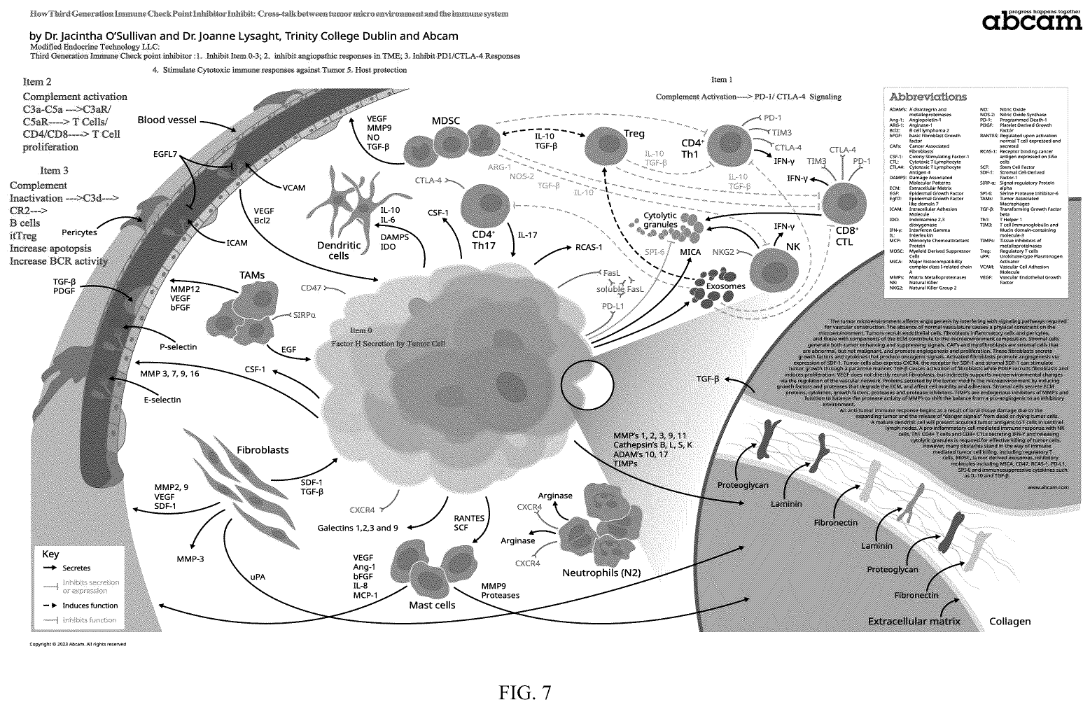

Fourth, down stream products that are generated due to breakdown of C3bBb, such as C3a-C5a, formation of C5b-C9 and Factor H downstream inactivated iC3b etc. contribute to stimulate T and B cell adaptive immune responses. Another representation of these processes is shown in FIG. 7 which is a diagram illustrating how third generation immune check point inhibitors limit cross-talk between tumor microenvironment and the immune system.

Thus, to generate host immunity, alternate complement system is the most ancient human defense system that has protected mankind from its most ancient time. It is this system that educates and primes early and delayed immune responses when threatened with intruders such as cancer cells.

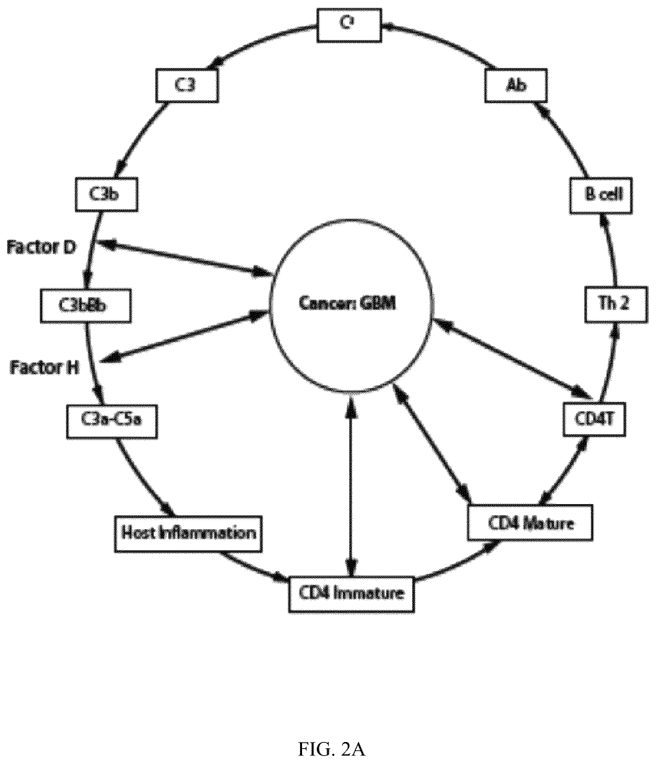

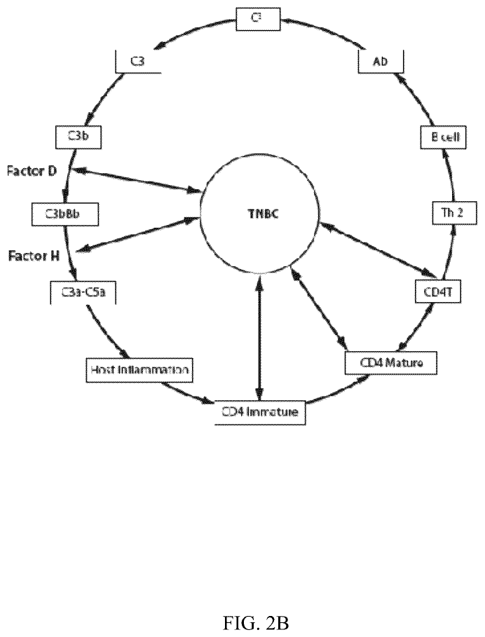

As shown in FIG. 2A, Glioblastoma is used as a reference example to show the impact of Factor H over secretion in cancer cells and its micro environment (TME) and how it hijacks the function of host immunity and turn it against itself to protect cancer cells, The survival of cancer and it's spread contributes to morbidity and mortality.

As shown in FIG. 2B, Triple Negative Breast Cancer (TNBC) is used as a further reference example to show the impact of Factor H over secretion in cancer cells and its micro environment (TME) and how it hijacks the function of host immunity and turn it against itself to protect cancer cells, The survival of cancer and it's spread contributes to morbidity and mortality. More specifically, the diagram shows C3 amplification based on immune pathogenesis in TNBC.

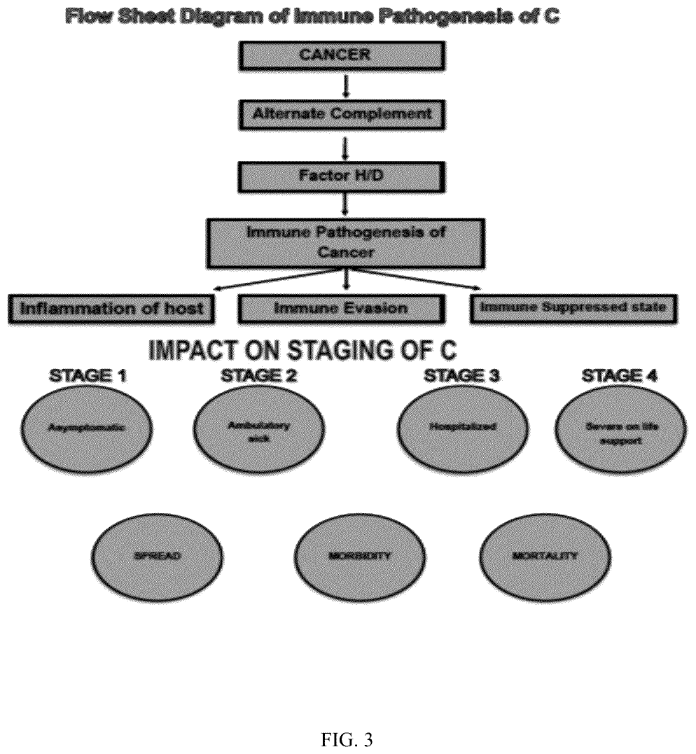

As shown in FIG. 3, AI approach is used to track detail sequences of Cancer immune pathogenesis and how it contributes to increase morbidity and mortality due to drug resistance and metastasis.

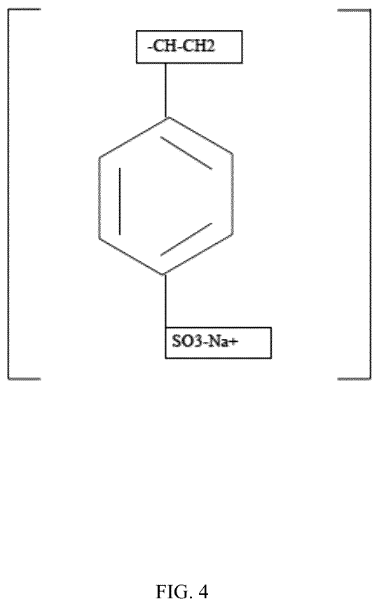

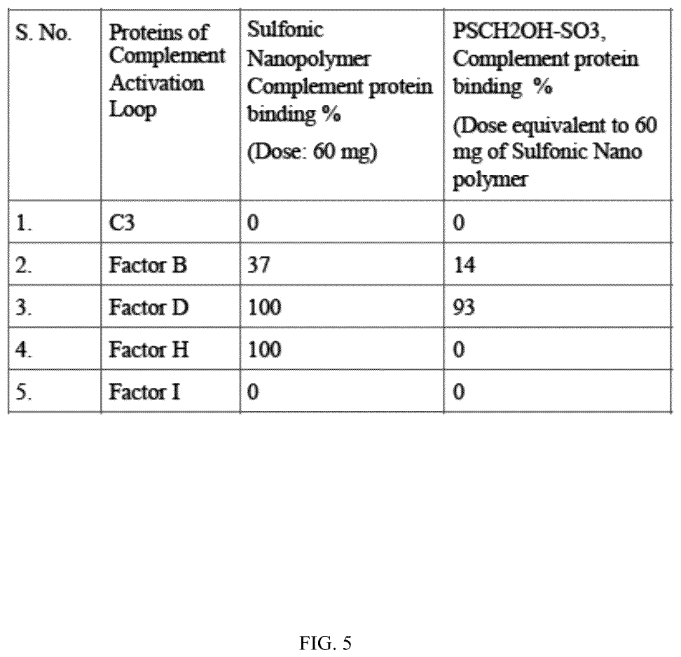

FIGS. 4 and 5 reflect AI based discovery of SNS Mimicking compound and its In-vitro data for controlled modulation of Factor B, D and H to down regulate C3 amplification loop.

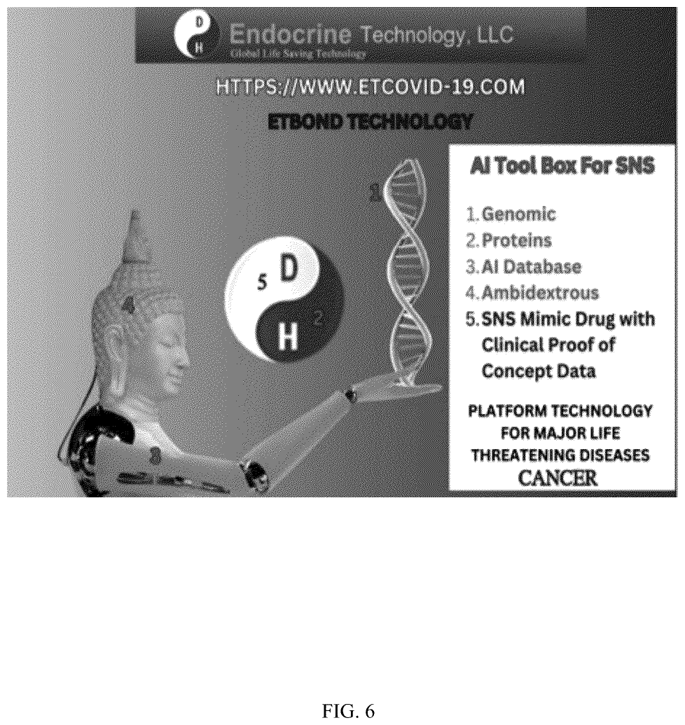

As shown in FIG. 6, The tool kit for AI has several elements 1-5 that need to be carefully coordinated and modified with strategic formulation methods to develop appropriate cancer therapy, prophylaxis and vaccine.

Thus, in item 1, focus is on Chromosome 1 at Q32 position. This is the weakest and most vulnerable location in entire genome of over 30,000 genes that contribute to Cancer survival, spread and its related morbidity and mortality.

Item 2, The key protein that is involved in above location that contribute to cancer survival, spread and its related morbidity and mortality is Factor H. When typed in Factor H and Cancer in Pub Med Central National Library of Medical Scientific Database, there are over 6946articles are found (see ncbi.nlm.nih.gov/pmc/?term=Factor+H+and+Cancer). A carefully analysis of this data reveal factor H is essential for fluid phase alternative pathway complement control, as well as for protection of host cells. Normal recognition of cell surfaces is required for effective protection. Thus, mutations and polymorphisms in factor H can result in inadequate protection and disease. Unwanted protection from factor H can result from efficient binding or secretion of Factor H by certain cancer cells. This contributes to increase cancer survival and spread with high morbidity and mortality.

Item 3: Futuristic AI application involves analysis of database of Factor H abnormalities in cancer and its relationship with patient characteristics to develop novel formulation methods for futuristic personalized and precision medicines for cancer therapy, prophylaxis and vaccines.

Item 4: Since the utility of Robotic model to deploy personalized consultancy for cancer depends upon the deep knowledge of medicine, recent advances in SNS modeling and industry efforts to develop novel drugs, machine learning is required to train AI model for predictive and personalized cancer therapy.

Item 5: SNS mimicking compound should ideally down regulate both Factor H and Factor D in a balanced way to avoid adverse effects of Factor H on host and should lead to successfully cytotoxic immune therapy.

For fastest deployment of cancer advances one need to consider individual patient's need in life science to expediently advance cancer therapy. FDA has several paths that can be used to develop compassion based approaches since SNS mimicking compound has human safety data.

From a Biotechnology perspective, AI modeling can be applied to predict human safety, appropriate formulation method to advance to phase 1-3 clinical trial to further determine safety and efficacy of futuristic therapies.

Reference examples of triple negative breast cancer and Glioblastoma Multiformes are presented, because of their resistance and lethal metastasis potentials, to outline the invention.

Glioblastoma multiformes (GBM) is the most common and deadly brain tumor that has average survival rate of 15 months in spite of aggressive surgery, radiation and chemotherapy (1). The incidence of GBM in the USA and Europe is 2-3 per 100,000 and therapeutic measures in this disease are classified under orphan disease affecting <200,000 persons in USA (one in every 1,500). This contributes to the escalation cost of treatment from $200,000.00 to $1,000,000 per patient. GBM disproportionately strikes elite members of society. Such as military personnel, sportsmen, actors, actresses, senators and high ranking politicians, musicians, radio, TV personalities, writers, etc. (2). One thought is that these are highly intellectual people driven by goal oriented activities. Their brain is always over activated and in some abnormal growth of brain occur resulting in GBM. We have applied artificial intelligence to develop Self-Non Self (SNS) modeling for this disease to simulate intricate aspects of immune pathogenesis of GBM and to explain drug resistance and metastasis. This is further extended with AI to develop novel drug that has SNS mimicking properties to target multifaceted immune pathogenesis of GBM that have AI predictability of human safety and efficacy. It is expected that it will improve safety, efficacy of treatment and prophylaxis of GBM. GT 103, is a Factor H antibody that has been recently developed to target over expression of Factor H in Glioblastoma and its related immune pathogenesis (3). This is used as a reference example to redefine immune pathogenesis of GBM through SNS modeling.

GT 103, is a Factor H antibody that has been developed to target over expression of Factor H in Glioblastoma and its related immune pathogenesis. A careful perusal of the article shows that targeting of Factor H with Factor H antibody is an inefficient way to treat GBM due to possible adverse effects of Factor H antibody, autoimmunity risk and risk of precipitating rare life threatening disorder such as (atypical Hemolytic Uremia Syndrome, aHUS). One possible novel approach is to use “Self-Non Self” (SNS) concept to detail sequential effects of immune pathogenesis of GBM leading to drug resistance and metastasis and then target it with SNS mimicking compound.

SNS Modeling: The recent advances in genomic and fundamentals of immunology pertain to Factor H. Factor H is a dominant immune regulatory molecule of alternate complement system that has protected mankind from the most ancient time through the control of “Self-Non Self” path. The gene sequence of this protein is located in chromosome 1 at Q32 position. There are over 30000 genes in human but genomic location of 1Q32 position for protein Factor H is a controller of “Self-Non Self Path” and there are over 17,000 published articles as of May 1, 2024 that details various characteristics of Factor H, its genetic mutations and abnormalities in various disease states that contribute to major life threatening diseases and how it could be targeted with Factor H antibody in preclinical models of different diseases. GB 103 is one such example that has been targeted for GBM therapy.

Immune pathogenesis of GBM: GBM over secretes Factor H. Its interactions in micro environment are detailed that contribute to immune pathogenesis of GBM and its TME. SNS modeling redefines immune pathogenesis and its sequential impact on drug resistance and metastasis. It is expected that by defining the multi-dimensional immune pathogenesis of cancer with host and its immune pathogenesis particularly with SNS modeling—will contribute to novel ways to overcome cancer resistance, metastasis and improve the safety profile of the patient. It is therefore expected that AI applications in describing the invention will allow predictive capabilities to judge safety and efficacy in cancer and will eliminate or overcome prior art problems of safety and efficacy in cancer therapy.

The end objective is to develop a better drug that mimic “SNS” modeling and provide predictable model of human safety and efficacy by deploying AI method of data analysis.

The Problem: Current cancer therapies ignore the new advances in fundamentals of immunity and genomics. These advances relate to:
1. Chromosome 1 at Q32 position and its protein Factor H
2. The interplay of Factor H in normal immunity, and
3. How it is compromised in various cancers.

Failure to consider above aspects in conventional cancer therapy has contributed to cancer resistance, metastasis and adverse drug reactions that has compounded the management problem of Cancer therapies.

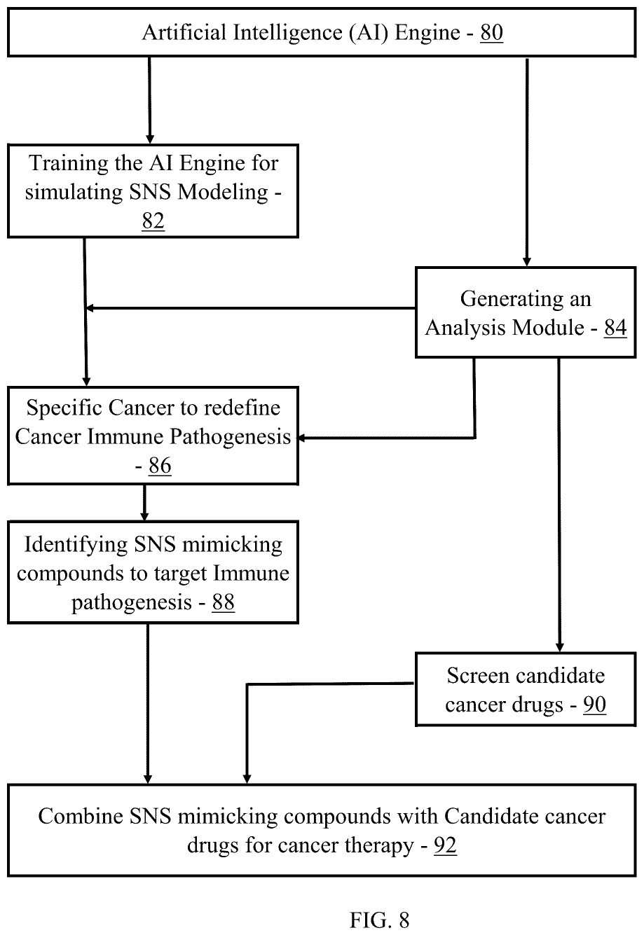

The Proposed Solution: FIG. 8

1. It outlines the general architectural plan to develop and apply Artificial intelligence to SNS modeling in both normal subjects and in cancer patients.

2. Above will help define Normal host immunity and how it is compromised by cancer. New Cancer immune pathogenesis is defined in terms of SNS Modeling.

3. Above understanding is further extended to identify key molecular targets in cancers such as Factor H, Factor B and Factor D.

4. Above targets modulate Self-Non Self Concept through C3 amplification loop in cancer patients.

5. SNS Mimicking compound is identified to intercept newly defined immune pathogenesis of cancer and labelled as third generation immune check point inhibitor.
a. First generation of immune check point inhibitor: Target PD/CT4 receptors and are related to adaptive immune dysfunction.
b. Second generation of immune check point inhibitor: Targets Factor H with Factor H monoclonal antibody that has adverse effects typically seen with any monoclonal antibodies.
c. Third generation of Immune check point inhibitor: Target Factor H, Factor B and Factor D that are specifically involved in C3 amplification loop. We carefully perused the immune pathogenesis of cancer and how it contributes to hyper activate C3 amplification loop to cause adverse effects in host, drug resistance and metastasis.

FIG. 8 depicts the processing by an Artificial intelligence engine that analyzes the data of SNS in a. Normal Subjects; b. In cancer subjects c. Identifies SNS mimicking compound that has human safety and efficacy data base and d. Develop novel formulation methods by combining SNS mimicking compound with cancer drugs including chemotherapeutic compounds, Monoclonal antibodies, complement modifying drugs and radiation therapies as well as cancer vaccines.

More specifically, FIG. 8 shows an Artificial Intelligence Engine in step 80. The AI Engine is trained for simulating SNS modeling in step 82. An analysis module of the AI engine generated in step 84 which directs the simulated SNS modeling to specific cancer to redefine cancer immune pathogenesis in step 86. The analysis module identifies SNS mimicking compounds to target immune pathogenesis of cancer in step 88. The analysis module is applied to screen candidate cancer drugs in step 90. The candidate cancer drugs are then strategically combined with the SNS mimicking compounds for cancer therapy in step 92.

At least one server, AI Engine 80 and/or Analysis module 84 may be designed and configured to receive training data. Training data consists of information containing correlations that a machine-learning process may use to model relationships between two or more categories of data elements. Training data may include a plurality of data points, data elements may be correlated by shared existence in a given data entry, by proximity in a given data entry, or data sets combining like data points or data elements. Multiple data entries in training data may be compared or contrasted to reveal trends or correlations between categories of data elements. Such trends or correlations could indicate a possible proportional or other mathematical relationship linking values belonging to the two categories. Multiple categories of data elements may be related in training data according to various correlations; correlations may indicate cause and effect relationships or predictive trends between categories of data elements. These relationships or trends can be used to model structures by machine-learning processes. Training data may be formatted and/or organized by categories of data elements, for instance by associating data elements with one or more descriptors corresponding to categories of data elements. As a non-limiting example, training data may include data entered in standardized forms or scanned or entered by natural language processing to extract data such that entry of a given data element in a given field in a form may be organized. Elements in training data may be linked to descriptors of categories by tags, tokens, or other data elements.

In an alternate embodiment of the invention, there is provided a system for identifying candidate drug compounds for treating cancer. The system includes a processor and a server for storing a multiplicity of scientific literature documents, genome data and a tangible, non-transitory computer readable medium storing instructions. When the instructions are executed, it causes the processor to generate an analysis module of an artificial intelligence engine which utilizes natural language processing to extract clinical data and relationships. The instructions further apply the analysis module to the scientific literature documents to analyze drug compounds with a relationship to the genome data to identify candidate drug compounds that effect a 1 q32 chromosome position.

In a further embodiment of the invention, there is provided a device for identifying candidate drug compounds for treating cancer. The device includes a processor and a server for storing a multiplicity of scientific literature documents, genome data. The device also includes an analysis module of an artificial intelligence engine which utilizes natural language processing to extract clinical data and relationships from the scientific literature. In addition, the device includes a comparison module to correlate the extracted clinical data from the analysis module to the genome data to identify candidate drug compounds that effect a 1 q32 chromosome position.

AI search led us to identify SNS mimicking compound that inhibit C3 amplification induced immune pathogenesis of GBM and its TME—A careful perusal of Recent Advances in Complement System suggest that Alternate Complement System is the key driver of Immune pathogenesis of GBM and its TME where there is hyper activation of proximal complement system. Factor D is a 25 kD fluid protein of Alternate Complement System. It is a key driver of proximal complement activation. The relentlessly activated proximal complement system cause down stream activation of inflammatory mediators that is key driver of GBM Related TME that contribute to drug resistance and metastasis.

Structural Formula of SNS mimicking Sulfonic Polymer that come with varying number of molecular size and properties such as N 1-100. Description: SNS Mimicking compound is sodium polystyrene sulfonate with variable N reflecting N-1 as monomer and N-100 as polymer with varying properties based on molecular weight.

It is benzene, diethenyl-polymer, with ethenylbenzene, sulfonated, sodium salt and has the structural formula as in Figure. polystyrene sulfonate (SPS), a cation-exchange resin prepared in the sodium phase with an in-vitro exchange capacity of approximately 3.1 mEq (in vivo approximately 1 mEq) of potassium per gram of the drug. The sodium content is approximately 100 mg (4.1 mEq) per gram of the drug. Polystyrene sulfonate (PSS) is a negatively charged polyelectrolyte bearing sulfonate groups. Due to the very low pKa of the sulfonate group (−2.1) PSS is fully charged in a wide range of pH and ionic strength conditions that contributes to its SNS binding with Factor H and Factor D.

Proposed Dose and the route of administration: Suspension of the drug should be freshly prepared and not stored beyond 24 hours. The average daily dose for GBM prophylaxis or for host immunity boosting effects is 250 mg twice a day. Each dose is administered as warm fluid such as 0.85% saline warmed to body temperature in 50-100 ml fluid. The fluid should be agitated gently to prevent precipitation.

### Contraindications

It is contraindicated in patients with hypokalemia or those patients who are hypersensitive to it or in extremely rare patients with Factor H Deficiency.

Drug Interactions Antacids: The simultaneous oral administration of ETBOND™ with non-absorbable cation-donating antacids and laxatives such as magnesium hydroxide and aluminum carbonate. This reaction generally occurs in significant way if given in extreme high dosage. In the recommended dose no interactions noted.

Digitalis: If the medicine is given in extreme large dosage, the toxic effects of digitalis on the heart, especially various ventricular arrhythmias and A-V nodal dissociation, are likely to be exaggerated by hypokalemia, even in the face of serum digoxin concentrations in the “normal”.

The binding characteristics of C3, Factor B, Factor D, Factor H and Factor I proteins were studied for sulfonic nano polymer and other polymers. NHS with buffer was used as normal control.

The dose of polymers used was standardized.

The NHS in (1:4) dilution with each of above polymers was incubated at 37 degrees C. in the water bath with gentle agitation for half hours to 1 hour durations in a series of experiments. The binding activity of complement proteins in the serum was analyzed in duplicate and mean result is depicted. Below are the key results.

### SNS Mimicking Compound: Sulfonic Polymers

In-Vitro Data for Factor D: A. The drug application is based on the fact that as shown above it is a complement inhibitor of Factor D that will inhibit relentlessly activated C3 amplification loop whose downstream products are responsible for C3a-C5a mediated inflammatory responses.

In-vitro Data for Factor H: The drug also inhibits Factor H. As shown above, Factor H is a dominant immune regulatory molecule of Alternate complement system that protect host in both at fluid and over cell membrane. Factor His 150 kD key immune regulatory protein of Alternate Complement System that is abundantly expressed in GBM Factor H and its downstream regulatory proteins, cause hyper activation uncontrolled activation of Factor D that is directed to host proteins in TME. This sets in repeated auto amplification of complement system contributing complement hyper inflammation and TME inflammation that progress to drug resistance and metastasis.

In vivo data in mice, it inhibits Complement system and CH 50 activity at 0.5 mg/kg body weight In-vivo Mice efficacy data in Inflammatory Disorders: Our initial model studied was Type 1 Non Obese Diabetic (NOD) mice as an autoimmune or inflammatory model of Type 1 Diabetes. This has been used successfully to inhibit inflammation in small animal models including Non Obese Diabetes Mice for the cure of diabetes. As compared to control 5-9 days, the inflammatory mediators were suppressed allowing prolongation of islet cell Graft for up to 80 days.

Currently there is no comparator therapy to modulate Factor H and Factor D function that has extensive human safety data. A number of drug companies are trying to develop such therapies as well as to target C3 amplification loop and are at preclinical levels.

### Drug Development Plan

Formulation strategy to reduce adverse effects and improve therapeutic efficacy is the part of drug development effort requiring immense input from scientists and international sources. Complexity of medical sciences, resistance to new ideas amongst experts, financial motives, legal complexity, lack of resources, and making life saving drug available at accessible prices are the critical issues in development of new therapeutic applications. Introduction of an established drug that is global, has proven safety, is in keeping with recent advances in “Genomics and fundamentals of immunology” and pertains to life saving application with extensive third party preclinical support allows us to have an AI strategy to over comes many of the hurdles that are inherent and draw attention of global experts as to what can be done to reduce complexity of medical care in cancer therapeutics.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, and systems according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention as set forth in the appended claims.

What is claimed:

1. A method for identifying candidate drug compounds for treating cancer comprising the steps of:
training an artificial intelligence engine for simulating Self-Non Self (SNS) modeling of normal subjects;
generating an analysis module of an artificial intelligence engine which directs the simulated SNS modeling to specific cancer to redefine cancer immune pathogenesis;
identifying SNS mimicking compounds with the analysis module to target immune pathogenesis of cancer; and
applying the analysis module to screen candidate cancer drugs that can be combined strategically with SNS mimicking compound for cancer therapy.

2. The method according to claim 1, wherein the method further includes the steps of:
storing genome data including data on over 30,000 human genes on a server.

3. The method according to claim 2, wherein the applying step includes applying the analysis module to the stored genome data with a preferential bias for references to the 1 q32 chromosome and its protein.

4. The method according to claim 2, wherein the applying step includes applying the analysis module to the stored genome data with a preferential bias for references to the 1 q32 chromosome and its protein Factor H as a weak and vulnerable genomic location within the genome data contributing to cancer immune pathogenesis.

5. The method according to claim 4, wherein the method further includes the steps of:
storing scientific literature documents on the server, wherein the scientific literature documents include over 17,000 articles giving details of SNS abnormalities in different cancers and wherein the simulating step includes using natural language processing to extract protein data for simulating SNS modeling of proteins.

6. The method according to claim 5, wherein SNS modeling of proteins are selected from the group consisting of Factor H, Factor D, and combinations thereof.

7. The method according to claim 6, wherein the storing step includes storing patient related characteristics including statistics, protocols and laboratory data,
wherein the identifying step includes providing an automated robotic system of a second artificial intelligence engine for identifying SNS compounds from the patient related characteristics to target immune pathogenesis of cancer.

8. The method according to claim 7, wherein the identifying step includes identifying SNS mimicking compounds to target immune pathogenesis of cancer for prophylaxis and therapy.

9. The method according to claim 8, wherein the candidate drug compounds include chemo therapy drugs, radiation, monoclonal antibodies, complement modifying drugs, vaccines and combinations thereof.

10. The method according to claim 9, wherein the applying step includes recursively applying the analysis module to develop updated formulation strategies covering current and evolving cancer therapies, chemo therapy drugs, radiation, monoclonal antibodies, complement modifying drugs and vaccines.
